(19) United States
(12) Patent Application Publication
(10) Pub. No.: US 2024/0074670 A1
(43) Pub. Date: Mar. 7, 2024

(54) NOVEL WALL SHEAR STRESS (WSS) ESTIMATION METHOD FOR 4D FLOW MRI

(71) Applicant: Purdue Research Foundation, West Lafayette, IN (US)

(72) Inventors: Pavlos Vlachos, West Lafayette, IN (US); Jiacheng Zhang, Brookfield, WI (US)

(21) Appl. No.: 18/223,157

(22) Filed: Jul. 18, 2023

Related U.S. Application Data
(60) Provisional application No. 63/396,806, filed on Aug. 10, 2022.

(51) Int. Cl. A61B 5/055; A61B 5/026; G01R 33/563

(52) U.S. Cl. CPC A61B 5/055; A61B 5/0263; G01R 33/5635; G01R 33/56366

(57) ABSTRACT

The invention generally relates to systems and methods that employ a novel wall shear stress (WSS) estimation method for 4D flow MRI. In certain embodiments, the invention provides systems and methods for determining Wall Shear Stress (WSS) with 4D flow Magnetic Resonance Imaging (MRI), that involve receiving 4D MRI flow data; calculating a velocity gradient (and optionally calculating the pressure field) from the 4D MRI flow data; correcting the velocity gradient (such as by using a spatial gradient of the pressure field to correct the velocity gradient), thereby producing a corrected velocity gradient; and determining a WSS from the corrected velocity gradient.

### RELATED APPLICATION

This Application claims the benefit of and priority to U.S. provisional patent application Ser. No. 63/396,806, filed Aug. 10, 2022, the content of which is incorporated by reference herein in its entirety.

### FIELD OF THE INVENTION

The invention generally relates to systems and methods that employ a novel wall shear stress (WSS) estimation method for 4D flow MRI.

### BACKGROUND

Vascular wall shear stress (WSS) is an important determinant of endothelial function and phenotype. The blood flow-induced WSS can cause morphological changes of endothelium and trigger biochemical and biological events, therefore leading to vascular remodeling and dysfunction. WSS has emerged as an essential feature of atherogenesis. The low WSS due to disturbed blood flow promotes atherogenesis, while high WSS is associated with plaque rupture. Abnormal WSS is also related to the growth and rupture of intracranial aneurysms. Additionally, WSS and WSS-derived metrics such as oscillatory shear index (OSI) are correlated with aortopathy. The distribution of low WSS and high OSI resembles the regions of aortic atherosclerotic lesions, and the abnormal WSS in the Bicuspid Aortic Valve (BAV) patients was associated with the aortic dilation. Therefore, the information on the magnitude, distribution, and variation of WSS can provide valuable insights for predicting and assessing vascular diseases.

WSS can be estimated from the velocity gradient at the vascular wall. 4D flow magnetic resonance imaging (MRI) resolves blood flow in space and time in vivo, enabling the estimation of WSS and WSS-derived metrics. Stalder et al. (“Quantitative 2D and 3D Phase Contrast MRI: Optimized Analysis of Blood Flow and Vessel Wall Parameters,” Magn. Reson. Med., vol. 60, pp. 1218-1231, 2008) introduced a method to evaluate the aortic WSS from the B-spline interpolation of the 4D flow velocity on manually positioned 2D planes. However, this method only resolves the WSS on the 2D slices, and the plane selection can be laborious. Several methods were introduced later to resolve the 3D WSS distribution on the vessel wall from the velocity profile along the wall-normal direction at each wall point. The method proposed by Potters et al. (“Volumetric arterial wall shear stress calculation based on cine phase contrast MRI,” J. Magn. Reson. Imaging, vol. 41, no. 2, pp. 505-516, 2015) uses smooth spline fitting of the velocity along the wall-normal direction and assumes no-slip boundary condition to evaluate the velocity gradients and WSS. The method has been applied to 4D flow data acquired in the aorta, carotid arteries, and intracranial aneurysms.

The accuracy of the WSS estimated from 4D flow data is affected by several factors, including the spatial resolution and segmentation. A significant inverse relationship was found between the estimated WSS and the spatial resolution of 4D flow data. The WSS estimated from in vivo 4D flow MRI was inconsistent with the results from high-resolution modalities, including computational fluid dynamics (CFD) and in vitro particle imaging velocimetry (PIV), potentially due to the limited resolution of MRI. The aortic WSS estimated from in vivo 4D flow data was 0-2 Pa, while patient-specific CFD models yielded a range of 0-30 Pa. The WSS estimated from 4D flow MRI was also lower than the CFD results in intracranial aneurysms and carotid bifurcations, and the differences were more significant in regions of higher WSS. Several multi-modality studies showed that the mean WSS evaluated from in vivo 4D flow MRI in the intracranial aneurysms was less than half of the results from patient-specific CFD simulations and in vitro PIV measurements. Because of the discrepancy in WSS magnitudes, the normalized parameters such as the normalized WSS and OSI are usually preferred for clinical and physiological investigations as they possess qualitatively similar distributions between MRI and other modalities.

### SUMMARY

The invention provides systems and methods that enhance the WSS estimation with 4D flow MRI. The inventive systems and methods (also referred to herein as pressure-gradient induced velocity-gradient correction (PG-VGC)), correct the velocity gradient based on the reconstructed pressure field gradient to improve the estimated WSS's accuracy. The conservation laws of mass and linear momentum are incorporated to formulate a linear system. This linear system is used to estimate the velocity-gradient errors with a least-squares approach. The error is then subtracted from the velocity gradient to improve the assessment of WSS. The systems and methods herein were first tested with synthetic 4D flow data of Womersley flow and flow in two cerebral aneurysms. The systems and methods were then applied to in vivo 4D flow data acquired in the cerebral aneurysms and aortas.

The performance of the systems and methods herein was compared to the state-of-the-art method based on smooth-spline fitting of velocity profile and the WSS calculated from uncorrected velocity gradient. The systems and methods of the invention improved the WSS accuracy by as much as 100% for the Womersley flow and reduced the underestimation of mean WSS by 39% to 50% for the synthetic aneurysmal flow. The approach was applied to in vivo 4D flow data acquired in cerebral aneurysms. The predicted mean WSS using the inventive approach was 31% to 50% higher than predictions using other prior art methods, and the distribution of high-WSS regions was consistent with patient-specific CFD results. The approach was further applied to in vivo 4D flow data in aortas. The mean WSS estimated using the inventive systems and methods was 4 to 6 times higher than WSS obtained with the other prior art methods. The range of WSS estimated using the systems and methods of the invention was consistent with previous CFD studies, and the predicted high-WSS regions showed an improved correlation with the vortical flow structures. The systems and methods improve the accuracy of WSS estimation from 4D flow MRI, which can help predict blood vessel remodelling and progression of cardiovascular diseases.

In certain aspects, the invention provides methods for determining Wall Shear Stress (WSS) with 4D flow Magnetic Resonance Imaging (MRI). The methods herein may involve receiving 4D MRI flow data; calculating a velocity gradient (and optionally calculating the pressure field) from the 4D MRI flow data; correcting the velocity gradient (such as by using a spatial gradient of the pressure field to correct the velocity gradient), thereby producing a corrected velocity gradient; and determining a WSS from the corrected velocity gradient.

In another aspect, the invention provides systems for determining Wall Shear Stress (WSS) with 4D flow Magnetic Resonance Imaging (MRI). The systems herein may include a processor configured to: receive 4D MRI flow data; calculate a velocity gradient (and optionally the pressure field) from the 4D MRI flow data; correct the velocity gradient (such as by using a spatial gradient of the pressure field to correct the velocity gradient), thereby producing a corrected velocity gradient; and determine a WSS from the corrected velocity gradient.

In certain embodiments of the systems and methods herein, the spatial gradient of the pressure field is employed to correct the velocity gradient based on the conservation of mass (COM) and conservation of linear momentum (COLM).

In certain embodiments of the systems and methods herein, the pressure field was calculated using a weighted approach with weights given as a function of physical and measurement variables:

w(s_wall)=w_min+(w_max−w_min) s_wall/s_(wall,max), with w_max/w_min=10;

where s_wall means distance from the blood vessel wall.

In certain embodiments of the systems and methods herein, the corrected velocity gradient was determined by subtracting velocity gradient errors (∇u=∇u_t+ϵ_∇u) estimated from:

COLM: ∇p/ρ=−∂u/∂t−u·(∇u−ϵ_∇u)+v∇·(∇u−ϵ_∇u)

COM: e·(∇u−ϵ_∇u)=0.

In certain embodiments of the systems and methods herein, the method uses the 4D MRI flow data in a whole region of interest (ROI). In certain embodiments of the systems and methods herein, the pressure field is reconstructed by integrating a pressure gradient estimated from a velocity and the velocity gradient in the whole ROI. In certain embodiments of the systems and methods herein, the reconstructed pressure field gradient is employed with additional regularization from a divergence-free constraint to correct the velocity gradient. In certain embodiments of the systems and methods herein, the WSS is estimated based on the corrected near-wall velocity gradient.

In certain embodiments of the systems and methods herein, the WSS is used to analyze physiological remodeling of a blood vessel wall. In certain embodiments of the systems and methods herein, results of the analysis of the physiological remodeling of a blood vessel wall provides an indication on growth and/or rupture of the blood vessel wall.

### BRIEF DESCRIPTION OF THE DRAWINGS

FIG. 1 panel A provides a flow chart of the WSS estimation procedure with PG-VGC method. FIG. 1 panel A provides a schematic demonstrates that the flow data in the whole region of interest (ROI) is used for enhancing the WSS estimation. The green box and red box indicate the data in the core-flow and near-wall regions, respectively.

FIG. 2 panel A shows the streamwise velocity and velocity gradient profiles for Womersley flow with different Womersley numbers (α). FIG. 2 panel B shows the time-dependent median and IQR of the streamwise pressure-gradient estimated at the wall points and in the core-flow region. Pgrad-u denotes the pressure gradient evaluated from the local velocity data, and Pgrad-p indicates the gradient of the reconstructed pressure field. FIG. 2 panel C shows the time-dependent median and IQR of the WSS estimated from different methods in one flow cycle.

FIG. 3 panels A-B show the relative RMSE of WSS estimated from synthetic Womersley 4D flow datasets with 0% noise (panel A) and 10% noise (panel B). The area of each wedge corresponds to the relative RMSE by each method, and the color scale indicates the comparison between the RMSE from Spline or Vgrads with PG-VGC.

FIG. 4 panels A-B show the velocity fields at peak systole of the BT (panel A) and ICA (panel B) aneurysms from CFD, synthetic MRI, and in vivo MRI.

FIG. 5 panel A shows the Bland-Altman plots comparing the WSS estimated from synthetic 4D flow data with the WSS from CFD, with the mean and standard deviation (std) of the WSS differences presented in the plots. FIG. 5 panel B shows The spatial distributions of the TAWSS estimated form the synthetic 4D flow data and from CFD.

FIG. 6 panel A shows the statistical distributions and mean values of the TAWSS estimated from in vivo 4D flow data of the BT aneurysm and the ICA aneurysm. FIG. 6 panel B shows the time-dependent median and IQR of the WSS estimated from the in vivo 4D flow data in a cardiac cycle. (c) The spatial distributions of the TAWSS estimated from the in vivo 4D flow data.

FIG. 7 panel A shows the statistical distributions and mean values of the TAWSS estimated from the in vivo aortic data. FIG. 7 panel B shows the time-dependent median and IQR of the estimated WSS in the cardiac cycle.

FIG. 8 panel A shows the velocity fields represented using 3D pathlines, the vortical structure indicated by iso-surfaces, the pressure distribution on the wall, and FIG. 8 panel B shows the relative WSS estimated by the three methods at peak systole from the in vivo 4D flow data of the three subjects. The green circles in the PG-VGC WSS distributions indicate the high WSS regions predicted by PG-VGC but are absent from the Spline or Vgrads estimations.

FIG. 9 is a high-level diagram showing the components of an exemplary data-processing system.

### DETAILED DESCRIPTION

The invention provides a novel wall shear stress (WSS) estimation system and method for 4D flow MRI. The system and method improves the WSS accuracy by using the reconstructed pressure gradient and incorporating the flow-physics constraints of the conservation of mass and linear momentum to correct the velocity gradient estimation. The approach was tested on synthetic 4D flow data of analytical Womersley flow and flow in cerebral aneurysms.

### Wall Shear Stress Estimation Algorithm

The WSS vector [symbol] at a wall point can be calculated as:

[symbol]

=2με̅· [symbol] , (1)

where μ is the dynamic viscosity of the blood, [symbol] ≡[nₓ n_{y} n_{z}]^{T} is the inward wall-normal vector with a magnitude of 1, and ε̅ is the deformation or strain rate tensor. The WSS value represents the magnitude of [symbol] in this study, and the time-averaged WSS (TAWSS) denotes the WSS averaged arithmetically over a cardiac cycle. The deformation tensor can be expressed by the velocity gradient tensor ∇̅u̅ and its transpose (∇̅u̅)^{T} as:

$$\overline{\overline{ε}} = \frac{1}{2} ( \overline{\overline{∇ u}} + {( \overline{\overline{∇ u}} )}^{T} ) . \qquad ( 2 )$$

The elements in ∇̅u̅ can be evaluated from the velocity field of the blood flow using numerical differentiation or interpolation, e.g., with smooth spline fitting or radial basis function (RBF) fitting.

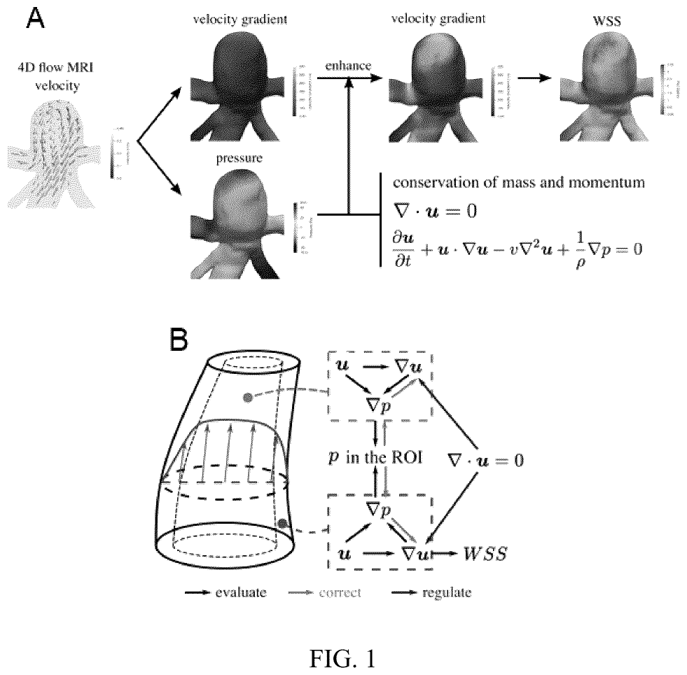

FIG. 1 panel A presents the WSS estimation procedure with PG-VGC. The velocity gradient and the pressure field are first calculated from the 4D flow data. The spatial gradient of the pressure field is employed to correct the velocity gradient based on the conservation of mass (COM) and conservation of linear momentum (COLM). The WSS is then determined from the corrected velocity-gradient. FIG. 1 panel B demonstrates that PG-VGC uses the data in the whole region of interest (ROI) defined as the region with blood flow for estimating WSS. The pressure field is reconstructed by integrating the pressure gradient estimated from the velocity and velocity gradient in the whole ROI. The reconstructed pressure field gradient is employed with additional regularization from the divergence-free constraint to correct the velocity gradients, and the WSS is estimated based on the corrected near-wall velocity gradients. The details of the algorithm are provided as follows:

(1) Pressure Reconstruction with Wall-Distance-Based Weighted Least-Squares

A Cartesian grid was constructed with each grid point corresponding to a voxel center of the 4D flow data. The blood flow region can be determined by segmenting the 4D flow image or the image from other modalities such as time of flight (TOF) angiography, and the surface representing the vessel wall can be created from the segmentation. The wall points where the WSS is of interest and the grid points corresponding to the voxels within the blood flow are combined to a list of N spatial points. The instantaneous pressure gradients at these spatial points were first estimated from the velocity field based on the COLM along each dimension as:

$${∇}_{i} p = - ρ ( \frac{\partial {u}_{i}}{\partial t} + u ∘ {G}_{x} {u}_{i} + v ∘ {G}_{y} {u}_{i} + w ∘ {G}_{z} {u}_{i} ) + μ {∇}^{2} {u}_{i} , \qquad ( 3 )$$

where the subscript i∈{x, y, z} indicates the spatial dimension. ∇ₓp, ∇_{y}p, and ∇_{z}p are the column vectors (∈ [symbol] ^{N}) of the pressure-gradient values at the spatial points. uₓ≡u, u_{y}≡v, and u_{z}≡w are the column vectors (∈ [symbol] ^{N}) of velocity data. Each velocity component is measured independently along each of the encoded direction with 4D flow MRI. ∘ represents the Hadamard (elementwise) product, and ρ is the fluid density. The temporal derivatives of velocity were calculated using the second order central (SOC) difference scheme. Gₓ, G_{y}, and G_{z} are the discrete gradient operators (matrices) with a size of N×N. The coefficients in the operators were determined using the RBF-generated finite difference method (RBF-FD), which is a meshless computational method based on the localized RBF-interpolant in a compact finite-difference mode. The discrete Laplacian operator was generated from the gradient operators as:

∇²=GₓGₓ+G_{y}G_{y}+G_{z}G_{z}. (4)

The pressure field in the whole ROI was reconstructed by spatially integrating the pressure gradients with weighted least-squares (WLS) as:

$${p}_{WLS} = {arg min}_{p} ( ‖ ( Gp - ∇ p ) ‖ ) , \qquad ( 5 )$$

$$with G = [ \begin{matrix}{G}_{χ} \\ {G}_{y} \\ {G}_{Z}\end{matrix} ] and ∇ p = [ \begin{matrix}{∇}_{x} p \\ {∇}_{y} p \\ {∇}_{z} p\end{matrix} ] ,$$

where p_{WLS}∈ [symbol] ^{N} is the column vector containing the reconstructed pressure at the spatial points, ∥·∥ represents the L2 norm, and the weight matrix [symbol] is a diagonal matrix with a size of 3N×3N. Each diagonal element of [symbol] corresponds to a spatial point and controls the influence of the pressure gradients at the point on the resulting pressure field. The weight was specified as:

$${d i a g}_{} = ( {max}_{} - {min}_{} ) \frac{s}{{s}_{max}} + {min}_{} , \qquad ( 6 )$$

with [symbol] ₘᵢₙ=1 and [symbol] ₘₐₓ=10,
where [symbol] _{diag}>0 is the diagonal element, s is the distance from the corresponding point to its closest wall point, sₘₐₓ is the maximum s in the ROI and corresponds to the radius of the largest artery in the ROI, and [symbol] ₘᵢₙ and [symbol] ₘₐₓ are the minimum and maximum weights, respectively. Equation (6) specifies the weights to increase linearly with the increase of the distance from the wall, therefore amplifying the effect of the core-flow pressure-gradient on the reconstructed pressure. It should be noted that the exact values of [symbol] ₘᵢₙ and [symbol] ₘₐₓ do not affect the pressure result if the ratio wₘₐₓ/wₘᵢₙ remains constant.

(2) Pressure-Gradient-Induced Velocity-Gradient Correction

The velocity gradient (∇̅u̅) evaluated from 4D flow data can be decomposed into a true component (∇̅u̅ₜᵣᵤₑ) and an error component (∇̅u̅ₑᵣᵣ) as:

∇̅u̅=∇̅u̅ₜᵣᵤₑ+∇̅u̅ₑᵣᵣ. (7)

∇̅u̅ₑᵣᵣ arises from the velocity measurement errors and gradient calculation, and affects the accuracy of WSS estimation. The COLM and COM can be expressed with the velocity gradient tensor as:

$$\frac{\partial \vec{u}}{\partial r} + \vec{u} \overline{\overline{∇ u}} = - v ∇ \cdot \overline{\overline{∇ u}} = + \frac{1}{ρ} ∇ p = 0 , \qquad ( 8 )$$

$$∇ \cdot \vec{u} ≡ \frac{\partial u}{\partial x} + \frac{\partial v}{\partial y} + \frac{\partial w}{\partial z} = \overline{\overline{I}} : \overline{\overline{∇ u}} = 0 , \qquad ( 9 )$$

where [symbol] =[u v w]^{T} is the flow velocity vector, p is the pressure, ∇ represents the gradient operator, “∇·” represents the divergence operator, and

$$v = \frac{μ}{ρ}$$

is the kinematic viscosity. I̅ᵢⱼ=δᵢⱼ is the identity tensor, and “:” is the double dot product such that I̅:∇̅u̅=δᵢⱼ∇̅u̅ᵢⱼ. The left-hand-side of (8) and (9) can be nonzero because of ∇̅u̅ₑᵣᵣ and the errors in [symbol] and ∇p. Assuming that ∇̅u̅ₑᵣᵣ is the major source of error, the equalities of (8) and (9) can be achieved by replacing ∇̅u̅ with ∇̅u̅ₜᵣᵤₑ, and the following equations can be subsequently derived from (8) and (9) by substituting ∇̅u̅ₜᵣᵤₑ for ∇̅u̅−∇̅u̅ₑᵣᵣ according to (7) as:

$$( \vec{u} - v ∇ ) \cdot {\overline{\overline{∇ u}}}_{err} = \frac{\partial \overline{u}}{\partial r} + \vec{u} \cdot ∇ u = - v ∇ \cdot ∇ u = + \frac{1}{ρ} ∇ p , \qquad ( 10 )$$

$$\overline{\overline{I}} : {\overline{\overline{∇ u}}}_{err} = \overline{\overline{I}} : \overline{\overline{∇ u}} . \qquad ( 11 )$$

Equations (10) and (11) relate ∇̅u̅ₑᵣᵣ to the residuals of (8) and (9), respectively, and can be used to estimate ∇̅u̅ₑᵣᵣ.
With the reconstructed pressure p_{WLS}, the following linear system can be constructed based on (8) for the COLM along each spatial dimension as:

$$\begin{matrix}[ u - {vG}_{x} & v - {vG}_{y} & w - {vG}_{z} ] [ \begin{matrix}{∇}_{x} {u}_{i , err} \\ {∇}_{y} {u}_{i , err} \\ {∇}_{z} {u}_{i , err}\end{matrix} ]\end{matrix} = \frac{\partial {u}_{i}}{\partial t} + u ∘ {G}_{x} {u}_{i} + v ∘ {G}_{y} {u}_{i} + w ∘ {G}_{z} {u}_{i} - v {∇}^{2} {u}_{i} + \frac{1}{ρ} {G}_{i} {p}_{WLS} , \qquad ( 12 )$$

where the subscript i∈{x, y, z} indicates the spatial dimension, i.e., uᵢ=u and Gᵢ=Gₓ if i=x. ∇ₓu_{i,err}∈ [symbol] ^{N} is the column vector containing the errors in

$$\frac{\partial {u}_{i}}{\partial x} ,$$

and this convention also applies to other velocity-gradient error terms in (12). The term Gᵢp_{WLS} is the spatial gradient of the reconstructed pressure. Three linear systems can be constructed from (12) for the COLM along x, y, and z dimensions. A linear system for the COM can be formulated based on (11) as:

$$[ \begin{matrix}I & I & I\end{matrix} ] [ \begin{matrix}{∇}_{x} {u}_{e r r} \\ {∇}_{y} {u}_{e r r} \\ {∇}_{z} {u}_{e r r}\end{matrix} ] = {G}_{x} u + {G}_{y} ν + {G}_{z} w , \qquad ( 13 )$$

where I represents the identity matrix with a size of N×N. The linear systems of (12) and (13) were combined to form a linear system with 4N equations and 9N unknown velocity-gradient errors. The combined linear system was solved with least-squares using the LSQR algorithm implemented in Python. The velocity gradients initially evaluated using the discrete gradient operators were corrected by subtracting the estimated velocity gradient errors, e.g.,

∇ₓu_{corr}=Gₓu−∇ₓuₑᵣᵣ, (14)

And the WSS was determined from the corrected velocity-gradient according to (1) and (2).

### Synthetic 4D Flow MRI Data of Womersley Flow

The proposed PG-VGC method's performance was first evaluated with synthetic 4D flow MRI data of Womersley flow. Womersley flow is representative of pulsatile flow in a circular pipe driven by an oscillatory pressure gradient and has been used to represent arterial flow in the cardiovascular system. The streamwise velocity component (w along z-direction) can be analytically expressed as:

$$w ( r , t ) = Real \{ {\sum }_{n = 0} \frac{i {P}_{{n}^{′}}}{ρ n ω} [ 1 - \frac{{J}_{0} ( α {n}^{1 / 2} {i}^{3 / 2} \frac{r}{R} )}{{J}_{0} ( α {n}^{1 / 2} {i}^{3 / 2} )} ] {e}^{in ωt} \} , \qquad ( 15 )$$

where r is the radial coordinate, R is the pipe radius, ω is the angular frequency of the first harmonic of the oscillatory pressure gradient

$$\frac{dp}{dz} , α = {R ( \frac{ω ρ}{μ} )}^{1 / 2}$$

represents the Womersley number, Pₙ′ is the pressure gradient magnitude for the harmonic at frequency nω, J₀(·) is the zeroth-order Bessel function of the first kind, i is the imaginary number, and Real{·} takes the real component of a complex number. The velocity components along other spatial dimensions are zero. The WSS can be determined analytically as

$${τ}_{w} ( t ) = Real \{ {\sum }_{n = 0} {P}_{{n}^{′}} \frac{R}{{Λ}_{n}} \frac{{J}_{1} ( {Λ}_{n} )}{{J}_{0} ( {Λ}_{n} )} {e}^{in ω t} \} , \qquad ( 16 )$$

with Λₙ=αn^{1/2}i^{3/2},
where J₁(·) is the first-order Bessel function of the first kind. The oscillatory pressure gradient was specified as:

$$\frac{dp}{dz} ( t ) = {P}_{0} sin ( 2 π t ) , \qquad ( 17 )$$

with ω=2π rad/s corresponding to a heart rate of 60 bpm. The velocity profile and WSS of the Womersley flow depend on the Womersley number, α. We considered the following α values to cover the typical range in the cardiovascular system: 1, 2, 4, 8, 12, 16, which lead to the following pipe diameters: 1.5, 3.1, 6.2, 12.4, 18.6, and 24.8 mm, with ρ=1100 kg/m³, μ=0.004 Pa·s, and ω=2π.

Synthetic 4D flow data were created from the analytical solution of the Womersley flow. The MRI signal corresponding to the streamwise velocity component was generated as:

$${M}_{w} = {I}_{mag} exp ( \frac{i π w}{venc} ) , \qquad ( 18 )$$

where M_{w} is the complex-valued MRI signal for w velocity, I_{mag} represents the signal magnitude, and venc is the velocity encoding sensitivity parameter. The value of I_{mag} was set as 1.0 in the flow (r<R) and 0.2 elsewhere, yielding a saturation ratio of 0.2. The venc was 1.5 times the maximum velocity in the flow field to avoid velocity aliasing. The synthetic 4D flow data were generated on a Cartesian grid in a 4R long pipe section. The voxel sizes (h) considered in this study were ½, ⅓, ¼, ⅕, 1/7, 1/9, 1/11, and 1/14 of the pipe diameter (D), and the temporal resolution (Δt) was 50 ms, yielding 20 frames per cycle. To mimic the spatial smoothing effect of 4D flow MRI, the MRI signal at each grid point was generated by convolving M_{w} with a sinc-function kernel [symbol] as:

$${M}_{w , MRI} = {M}_{w} * 𝒦 , with 𝒦 ( x , y , z ) = sin c ( \frac{x}{h} ) sin c ( \frac{y}{h} ) sin c ( \frac{z}{h} ) , \qquad ( 19 )$$

where * denotes the convolution operation. The sinc-function kernel has been employed to simulate the spatial blurring of Cartesian 4D flow MRI due to limited k-space coverage in previous studies. The MRI signals for the other velocity components (M_{u,MRI} and M_{v,MRI}) were created similar to (19). According to a four-point acquisition method, the reference MRI signal (M_{0,MRI}) was created from a zero velocity field such that the phase difference between the flow-sensitive signal and M_{0,MRI} matched the corresponding velocity component. To consider the measurement noise, normally distributed noise (ϵ_{MRI}) was added to the complex-valued MRI signal. This complex noise is defined as:

ϵ_{MRI}=ϵ_{RE}+iϵ_{IM}, (20)

with ϵ_{Re}, ϵ_{Im}∈ [symbol] (0, σ²),
where ϵ_{RE} and ϵ_{IM} indicate the noise added to the real and imaginary parts, respectively. The standard deviation σ was set based on the predefined velocity-to-noise ratio (VNR) as:

$$σ = \frac{{I}_{mag}}{VNR} \frac{π}{\sqrt{2}} \frac{\overline{V}}{venc} \qquad ( 21 )$$

where V̅ is the mean velocity in the flow field. The 4D flow velocity data was then obtained from the MRI signal, e.g., for the streamwise component as:

$${W}_{MRI} = \frac{venc}{π} {ψ}_{w} = \frac{venc}{π} angle ( {M}_{w , MRI} {M}_{0 , MRI}^{*} ) , \qquad ( 22 )$$

where ψ_{w} is the phase difference, M*_{0,MRI} is the complex conjugate of M_{0,MRI}, and angle(·) evaluates the angle of the complex number. For each Womersley number and spatial resolution, one dataset without noise and one dataset with a VNR of 10 (10% noise) were created. For each dataset, 100 wall points were selected for the WSS evaluation and analysis.

### Aneurysmal Flow Acquisition and Simulation

To test the proposed PG-VGC method with physiological flows, in vivo 4D flow MRI data were acquired in a basilar tip (BT) aneurysm at San Francisco VA Medical Center and an internal carotid artery (ICA) aneurysm at Northwestern Memorial Hospital with a 3T MRI scanner (Skyra, Siemens Healthcare, Erlangen, Germany). The 4D flow data were on Cartesian grids with the spatial resolution of 1.25×1.25×1.33 mm³ for the BT aneurysm and 1.09×1.09×1.30 mm³ for the ICA aneurysm. The temporal resolution was 40.5 ms (20 frames per cycle) and 44.8 ms (13 frames per cycle) for the BT and ICA aneurysms, respectively. The contrast-enhanced magnetic resonance angiography (CE-MRA) data was also acquired for the BT aneurysm with the spatial resolution of 0.7×0.7×0.7 mm³. For the ICA aneurysm, non-contrast time of flight (TOF) angiography was acquired with a spatial resolution of 0.4×0.4×0.6 mm³. The CE-MRA and TOF images were segmented to create surfaces (STL) of the vessel wall. The wall points and wall-normal extracted from the STL surfaces were used for evaluating and analyzing the WSS. Approval of all ethical procedures and protocols was granted by the institutional review boards (IRB) at Purdue University, Northwestern Memorial Hospital, and San Francisco VA Medical Center.

Additionally, patient-specific CFD simulations were performed using FLUENT 18.1 (ANSYS) with the created surfaces and the flow waveforms obtained from 4D flow data as the inflow and outflow boundary conditions. The flow was assumed to be laminar, incompressible, and Newtonian. The walls of the vessel were assumed to be rigid. The density and dynamic viscosity used for the simulations were 1060 kg/m³ and 0.0035 Pa·s. More details on the in vivo imaging and CFD simulations can be found in Brindise et al. (“Multi-modality cerebral aneurysm haemodynamic analysis: in vivo 4D flow MRI, in vitro volumetric particle velocimetry and in silico computational fluid dynamics,” J. R. Soc. Interface, vol. 16, no. 158, p. 20190465, September 2019). Synthetic 4D flow datasets were also created based on the results from the patient-specific CFD simulations using the approach described in Section II-B with the same spatial and temporal resolutions as the in vivo data and a VNR of 10 (10% noise).

### In Vivo Aortic 4D Flow MRI

In vivo 4D flow data were acquired in the aortas from three subjects to evaluate the performance of PG-VGC, including a patient with bicuspid aortic valve (BAV), a patient with tricuspid aortic valve and an aortic aneurysm (TAV-AA), and a health control subject with tricuspid aortic valve. The scans were performed in a sagittal oblique volume on a 1.5T scanner (MAGNETOM Avanto, Aera, Siemens, Erlangen, Germany) at Northwestern Memorial Hospital with prospective ECG gating and during free breathing. Gadolinium-based contrast (Magnevist, Ablavar, or Gadavist) were used for imaging the two patients, while no contrast was used on the control subject. The resolutions and scan parameters were presented in Table I. The venc was 150 cm/s for the TAV-AA and control scans and 175 cm/s for the BAV scan. No velocity aliasing was observed. The patient data for this IRB approved study were retrospectively included with waiver of consent. The healthy control subject underwent a research cardiac MRI after written informed consent was obtained from the study participant. A static mask of the blood vessel was created for each dataset based on the magnitude image and the time-averaged velocity magnitude, which was manually corrected by an expert observer using Mimics (Materialise NV, Belgium). A smooth surface (STL) was then generated from the mask to represent the vessel walls. The wall points and wall-normal from the surfaces were used for estimating the WSS.

TABLE I
The spatial and temporal resolutions, the number of cardiac
timeframes (Nₜᵢₘₑ), the flip angle, echo time (TE), and
repetition time (TR) for the in vivo aortic scans

| | | | | Flip | |
|---|---|---|---|---|---|
| | Voxel size | Δt | | angle | TE/TR |
| Subject | (mm³) | (ms) | Nₜᵢₘₑ | (°) | (ms) |
| TAV- | 2.375 × 2.375 × 3 | 37.6 | 21 | 15 | 2.3/4.7 |
| AA | | | | | |
| BAV | 2.125 × 2.125 × 2.5 | 38.4 | 24 | 15 | 2.4/4.8 |
| Control | 2.375 × 2.375 × 2.4 | 38.4 | 21 | 7 | 2.5/4.8 |

### Performance Evaluation and WSS Error Analysis

The proposed method's performance was evaluated by assessing the accuracy of the WSS estimated from the synthetic 4D flow data of the Womersley flow and the cerebral aneurysmal flow. The WSS from the analytical solution was employed as the “ground truth” for the Womersley flow. The WSS error level of each test case was represented by the root-mean-square error (RMSE) evaluated as:

$$RMSE = \sqrt{\frac{{\sum }_{i = 1}^{{N}_{t}} {\sum }_{j = 1}^{{N}_{wall}} {( {WSS}_{i , j} - W S {S}_{true , i , j} )}^{2}}{{N}_{t} \times {N}_{w a l l}}} , \qquad ( 23 )$$

where Nₜ and N_{wall} represent the number of timeframes and the number of wall points, respectively. The relative RMSE was determined as the RMSE normalized by the root-mean-square of the ground truth WSS. For the synthetic aneurysmal 4D flow data created based on the CFD results, the WSS obtained from the CFD simulations was considered as the “ground truth”. To demonstrate the improvement by PG-VGC, the state-of-the-art method introduced by Potters et al. was employed in this study and referred to as “Spline” since it evaluates the WSS with the smooth-spline fitting of the velocity profile. WSS was also estimated using (1) and (2) from the uncorrected velocity gradients and was referred to as “Vgrads”. The accuracy of Spline and Vgrads was also assessed and compared to the PG-VGC method.

### Synthetic 4D Flow Data of Womersley Flow

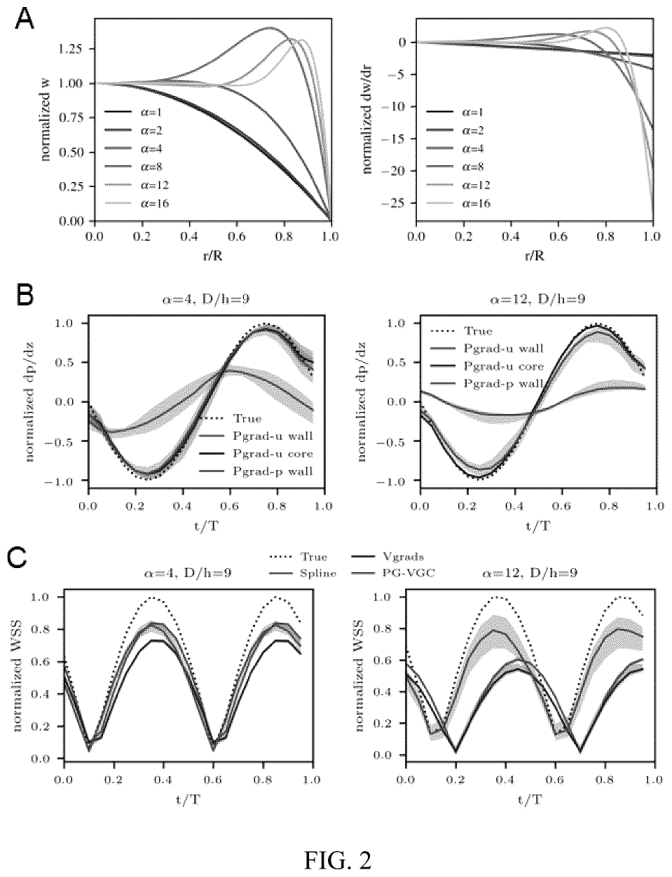

FIG. 2 panel A presents the normalized streamwise velocity profile and the velocity gradient with respect to radius (dw/dr) at the peak flow rate from the analytical solutions of the Womersley flow with different Womersley number (α). The velocity was normalized by the centerline velocity (w_{center}), and the velocity gradient was normalized by w_{center}/R. At low α (1 and 2), the velocity profile was close to parabolic, and the velocity gradient was relatively low. With an increase of α, the shear layer became thinner leading to a steeper velocity gradient near the wall. FIG. 2 panel B shows the time-dependent median and interquartile range (IQR) of the streamwise pressure gradient (dp/dz) evaluated at the wall points and in the core-flow region with r<0.5R from the flow data with different α and the spatial resolution of D/9. The pressure gradient directly evaluated from velocity field with (3) is denoted as “Pgrad-u”, while “Pgrad-p” represents the gradient of the reconstructed pressure field. The pressure gradients were normalized by the amplitude of the “true” dp/dz from the analytical solution. For both datasets, the Pgrad-u in the core-flow matched the true dp/dz with errors less than 0.1 for most timeframes, while the Pgrad-u at the wall deviated from the true solution by as much as 0.5 for α=4 and up to 0.8 for α=12. The Pgrad-p at the wall achieved similar accuracy as the Pgrad-u in the core-flow and was more reliable than the Pgrad-u at the wall. FIG. 2 panel C compares the WSS estimated using different methods with the “ground truth” from the analytical solution, and the WSS was normalized by the amplitude of the true WSS. The Spline and PG-VGC methods estimated similar WSS results at α=4 with errors less than 0.2 and were more accurate than the WSS by Vgrads with errors up to 0.3. From the dataset with α=12, Spline and Vgrads underestimated the amplitude of WSS by 40% with a phase shift of approximately 0.1 s, while PG-VGC underpredicted the WSS amplitude by 20%.

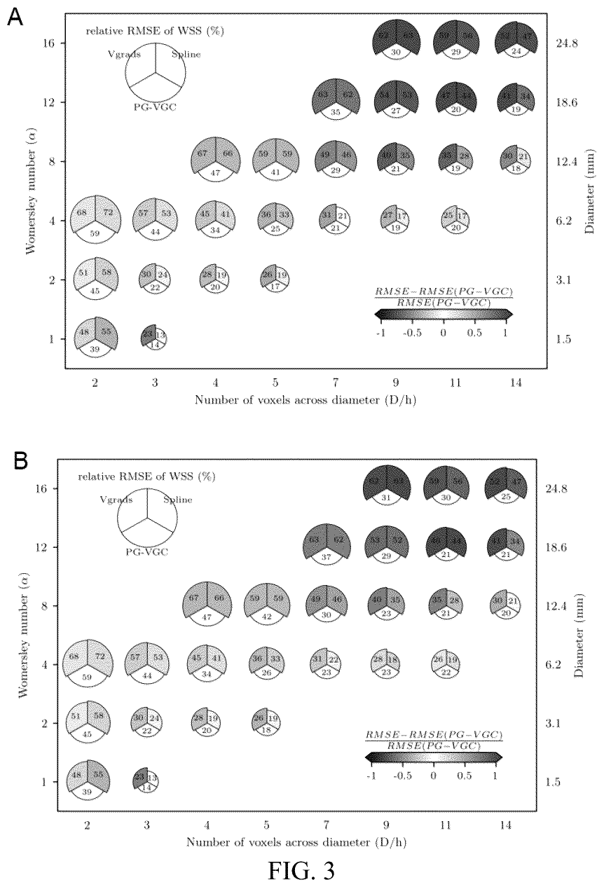

FIG. 3 panels A-B presents the relative RMSE of the WSS estimated using different methods as compared to the ground truth from the synthetic Womersley flow datasets with different α, grid resolution, and noise level. The cases with a grid resolution less than 0.5 mm or greater than 3.5 mm were uncommon in practical applications and were excluded from the analysis. The wedge area corresponds to the relative RMSE of each method, and the color on the wedge indicates the RMSE by Spline or Vgrads as compared to PG-VGC. A red wedge suggests higher RMSE by Spline or Vgrads than PG-VGC, while a blue wedge indicates that the WSS estimated by Spline or Vgrads were more accurate than PG-VGC. Compared to Spline and Vgrads, the proposed PG-VGC method yielded more accurate WSS for most datasets. Greater improvement was achieved by PG-VGC for higher a cases with more than 100% improvement at α of 12 and 16. The estimated WSS's accuracy was affected by the resolution and α. With an α of 8 and without noise, the relative RMSE by PG-VGC reduced from 47% to 15% as the resolution increased from 4 to 14 voxels across the diameter, and the relative RMSE of Spline and Vgrads changed from 66% to 21% and 67% to 30%, respectively. With 9 voxels across the pipe diameter, the relative RMSE of PG-VGC increased from 19% to 30% as α increased from 4 to 16, while the relative RMSEs by Spline and Vgrads were 17% to 63% and 27% to 62%, respectively. The 10% noise led to slightly increased RMSE for several cases with relatively fine resolution as compared to the results without noise.

### Synthetic and In Vivo Aneurysmal 4D Flow

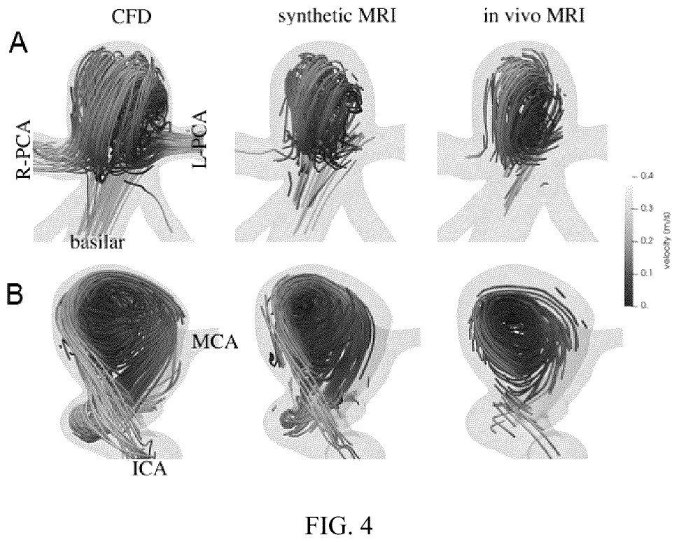

The velocity fields at peak systole from the patient-specific CFD simulations, the synthetic 4D flow data, and the in vivo 4D flow data were shown in FIG. 4 panels A-B for the BT and ICA aneurysms, respectively. For the BT aneurysm, the flow entered from the basilar artery, circulated in the aneurysmal sac, and then exited primarily through the posterior cerebral arteries (PCAs). For the ICA aneurysm, the flow entered from the ICA, circulated in the aneurysmal sac, and exited through the distal ICA and the middle cerebral artery (MCA). The inflow jet in the ICA aneurysm from the CFD data and synthetic MRI data reached the tip of the sac at peak systole with a maximum velocity of approximately 0.5 m/s. In comparison, the inflow jet from the in vivo MRI had a shorter penetration into the aneurysm and a lower maximum velocity at 0.3 m/s. There were 5-6 MRI voxels across the diameter of the BT aneurysmal sac, while 8-10 voxels were located along the diameter of the ICA aneurysmal sac. Compared to CFD, the flow structures in the near-wall regions and in smaller vessels were under-resolved in the MRI data.

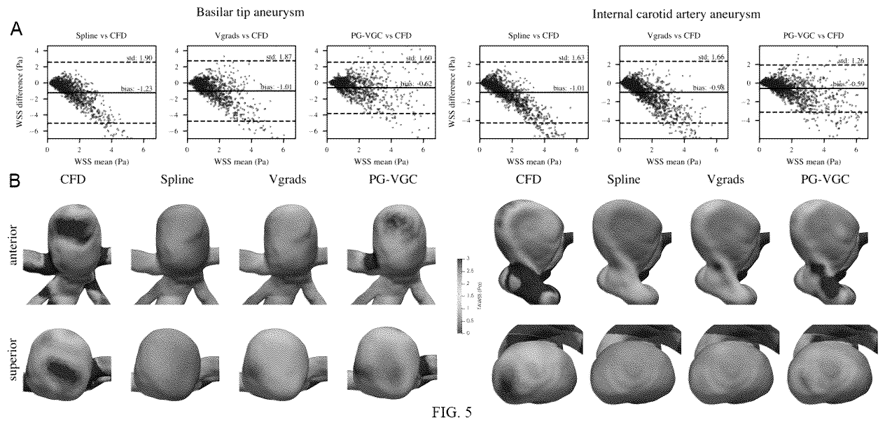

The Bland-Altman plots in FIG. 5 panel A compare the estimated WSS from the synthetic 4D flow data with the CFD WSS values serving as the “ground truth”. All three methods underestimated the mean of the WSS differences. Compared to the CFD WSS with a mean of 2.1 Pa, the WSS bias was −1.23 Pa for Spline, −1.01 Pa for Vgrads, and −0.62 Pa for PG-VGC in the BT aneurysm. The WSS bias was −1.01 Pa for Spline, −0.98 Pa for Vgrads, and −0.59 Pa for PG-VGC in the ICA aneurysm with a mean CFD WSS of 1.9 Pa. The PG-VGC method reduced the WSS underestimation by 39% to 50% and improved the robustness compared to the other methods, as suggested by the lower bias values and standard deviations. FIG. 5 panel B compares the spatial distributions of the TAWSS from CFD and synthetic MRI. High WSS regions were observed in the anterior view and superior view of the BT aneurysmal sac in CFD results, which PG-VGC also predicted, but absent in the results obtained by Spline and Vgrads. For the ICA aneurysm, the PG-VGC method predicted the high WSS region at the tip of the aneurysmal sac from the superior view, which was missing from Spline and Vgrads results. Moreover, PG-VGC yielded higher WSS in the ICA than Spline and Vgrads, which was more consistent with CFD.

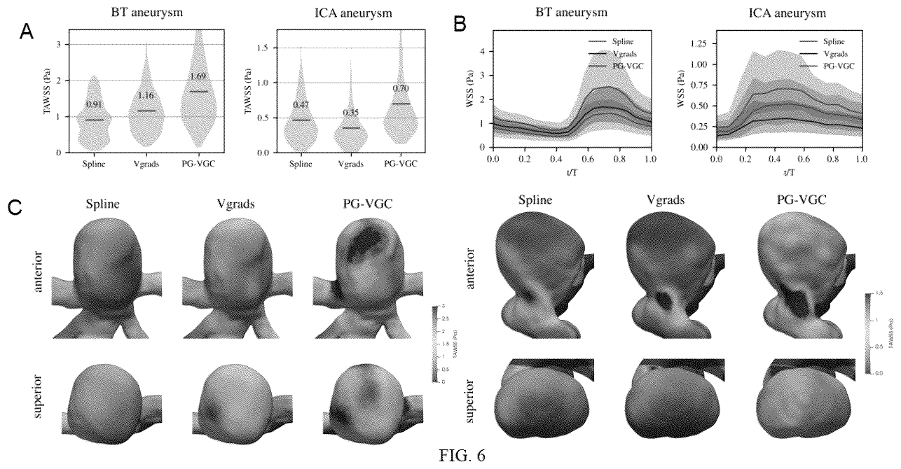

FIG. 6 panel A compares the statistical distributions of the TAWSS estimated from the in vivo aneurysmal data. The mean TAWSS estimated by Spline was 46% and 33% lower than that estimated by PG-VGC for the BT and ICA aneurysm, respectively, while the mean TAWSS obtained by Vgrads was 31% and 50% lower than that from PG-VGC for the BT and ICA aneurysm respectively. FIG. 6 panel B presents the time-dependent median and IQR of the estimated WSS for the two aneurysms. The different methods obtained similar WSS waveforms; however, PG-VGC predicted higher WSS than the other methods at all timeframes. FIG. 6 panel C shows the spatial distributions of the estimated TAWSS. PG-VGC predicted additional high WSS regions in the BT aneurysmal sac as shown on the anterior and superior views. The three methods yielded qualitatively similar TAWSS distributions for the ICA aneurysm, with higher WSS predicted by PG-VGC than the other two methods around the “neck” of the aneurysmal sac.

### In Vivo Aortic 4D Flow MRI

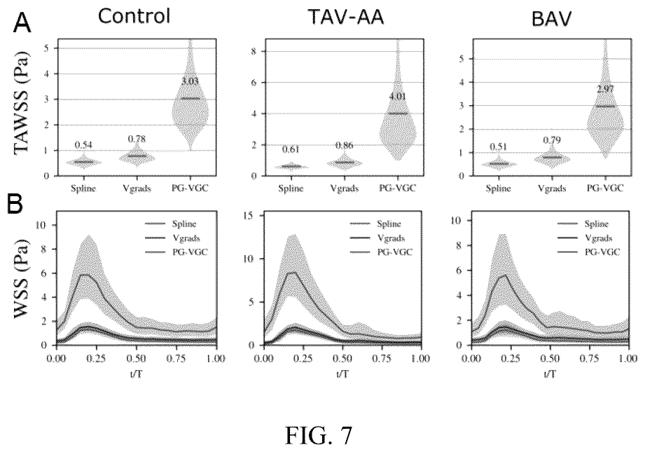

FIG. 7 panel A compares the statistical distributions of the TAWSS estimated from the in vivo aortic data of the three subjects. The mean TAWSS by PG-VGC was 5.6 to 6.6 times the mean TAWSS assessed by Spline and 3.7 to 4.7 times the mean TAWSS assessed by Vgrads. Similar profiles of the time-dependent median of WSS were obtained from all methods as shown in FIG. 7 panel B, while PG-VGC predicted higher WSS and larger IQR for all the timeframes. The median WSS estimated at peak systole was 5 to 7 Pa by PG-VGC and 1 to 2 Pa by Spline and Vgrads.

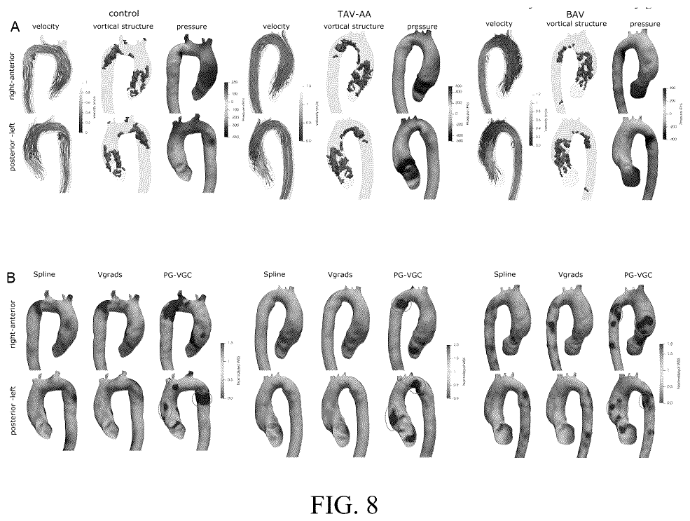

FIG. 8 panels A-B present the velocity field, the vortical structure (VS), the pressure distribution on the wall, and the relative WSS. The relative WSS is defined as the WSS normalized by its global average at peak systole for the three subjects. The velocity fields are represented using 3D pathlines whose color corresponds to the velocity magnitude. The VSs are included because it has shown to be correlated with high WSS in the aorta. The VSs were detected using the Q-criterion and are represented by the iso-surfaces in FIG. 8 panel A. Both the right-anterior (R-A) and the posterior-left (P-L) views were presented. The green circles in the PG-VGC WSS distributions indicate the high WSS regions predicted by PG-VGC but are absent from the Spline or Vgrads estimations. For all subjects, VSs were observed in the ascending aorta near the aortic root and in the descending aorta near the aortic arch, with a corresponding location of the local minimum pressure. For the control subject, high WSS regions around the VSs were calculated by all three methods, as shown on the R-A view. However, only PG-VGC predicted the marked high WSS regions on the P-L view. For the TAV-AA subject, PG-VGC predicted the high WSS region in the aneurysm at the aortic arch, which was missing from the results obtained with Spline and Vgrads. PG-VGC also yielded the high WSS region next to the VS in the ascending aorta near the P-L wall. For the BAV subject, PG-VGC predicted multiple regions with high WSS in the ascending aorta corresponding to the VSs near the R-A and P-L wall, which were not seen in the Spline or Vgrads results.

### Analysis

The invention herein introduced, evaluated, and applied a method for WSS estimation from 4D flow MRI. The systems and methods of the invention improve the WSS estimation using the flow data in the whole ROI to enhance the near-wall velocity gradients. The near-wall velocity-gradient calculation from 4D flow data is commonly unreliable because of the systematic velocity errors caused by the partial-volume effects and intravoxel phase dispersion. Moreover, the velocity gradient near the wall is typically higher than the core-flow region, as shown in FIG. 2 panel A, therefore increasing the bias error in the gradient calculation in cases with limited spatial resolution. The uncertainty in the wall location also affects the velocity-gradient's accuracy if the wall points are incorporated in the gradient evaluation. The velocity-gradient correction by PG-VGC was based on the flow physics constraints of COLM and COM. At the stationary wall, the COLM is reduced to the balance between the pressure force and the viscous diffusion and directly relates the velocity gradient to the pressure gradient. The near-wall pressure gradient evaluated using (3) based on the near-wall velocity gradient can be unreliable, while the pressure gradient estimated in the core-flow is more accurate as shown in FIG. 2(b). We reconstructed the pressure field in the ROI by spatially integrating the pressure-gradients using WLS. This WLS approach is a global optimization process, dominated by the pressure-gradients in the core-flow regions due to their greater weights. The spatial gradient of the reconstructed pressure in the near-wall region is more reliable than the uncorrected pressure-gradient as shown by comparing Pgrad-u and Pgrad-p in FIG. 2 panel B. Therefore, the more reliable Pgrad-p was used and proved to be effective for correcting the near-wall velocity-gradient. The COM's effect on the velocity-gradient correction is expected to be secondary to COLM as it only affects 3 of the 9 elements in ∇̅u̅.

The PG-VGC method improves the mean and the range of WSS estimated from 4D flow data compared to other methods. A previous study has shown that the WSS estimated from phase-contrast MRI data in intracranial aneurysms depended on the spatial resolution with 50 to 60% underestimation of the mean WSS at a resolution of 1 mm. The PG-VGC reduced the underestimation of the mean WSS in the synthetic aneurysmal flows by 39-50% compared to Spline and Vgrads methods. The mean WSS predicted by PG-VGC from the in vivo aneurysm flow measurements was 31 to 50% higher than that predicted by Spline and Vgrads. Therefore, the increased WSS predicted by PG-VGC improved the accuracy of the WSS estimation in cerebral aneurysms. For the in vivo aortic data, Spline and Vgrads yielded a median WSS of 1 to 2 Pa at peak systole, which was consistent with the results in previous studies using similar methods. However, the common range of mean aortic WSS at peak systole was 5 to 20 Pa according to CFD studies. The underestimation of WSS in the aorta with 4D flow MRI was due to the low spatial resolution of the imaging data. Perinajová et al. (“Assessment of turbulent blood flow and wall shear stress in aortic coarctation using image-based simulations,” Biomed. Eng. Online, vol. 20, no. 1, pp. 1-20, 2021) estimated the WSS from spatially down-sampled CFD data in a flow phantom of aortic coarctation, and the mean WSS was underestimated by 34% at a resolution of 0.2 mm and by 63% at a resolution of 0.7 mm. In the present study, the spatial resolution of in vivo aortic MRI data was 2-3 mm, which was expected to cause greater WSS underestimation compared to higher resolutions. PG-VGC predicted 4 to 6 times higher mean WSS than Spline and Vgrads resulting in better agreement with the results from previous CFD studies. The overall increase of the WSS magnitude computed by PG-VGC can potentially resolve the inconsistency between the WSS obtained from different modalities as observed in previous studies. The improvements achieved by PG-VGC method promote the use of WSS in addition to the normalized parameters such as relative WSS and OSI for the investigation of WSS-related cardiovascular diseases with 4D flow MRI.

The PG-VGC method also improves the prediction of the relative WSS distribution. From the synthetic aneurysmal flow data, PG-VGC recovered the high WSS regions absent in the Spline and Vgrads results, as shown in FIG. 5 panel B. PG-VGC also predicted additional high WSS regions for the in vivo BT aneurysm data that were absent in results computed with the other methods as shown in FIG. 6 panel C, thus obtaining WSS distribution more consistent with the CFD results shown FIG. 5 panel B. Based on in vivo aortic 4D flow data, PG-VGC predicted high WSS in regions corresponding to near the VSs, which is consistent with the previous finding that high WSS correlates with VSs in the aorta. The Spline and Vgrads methods failed to predict several of these high WSS regions, as highlighted in FIG. 8 panels A-B. The improved prediction of relative WSS distribution by PG-VGC is valuable for detecting regions with abnormal WSS, as these are related to cardiovascular disease progression such as growth and rupture of intracranial and aortic aneurysms.

The data herein show that the invention provides a novel WSS estimation method for 4D flow MRI. The method uses the pressure gradient estimated from the flow data in the whole ROI and flow physics constraints to correct the velocity gradient, therefore enhancing the WSS estimation. The method's performance was evaluated using synthetic and in vivo 4D flow data in cerebral aneurysms and thoracic aortas. The proposed method showed reliable estimation of the mean and the relative distribution of WSS with as much as 100% improvement in WSS accuracy. The method can benefit clinical applications of 4D flow MRI as it improves the accuracy of the WSS estimation.

### System Architecture

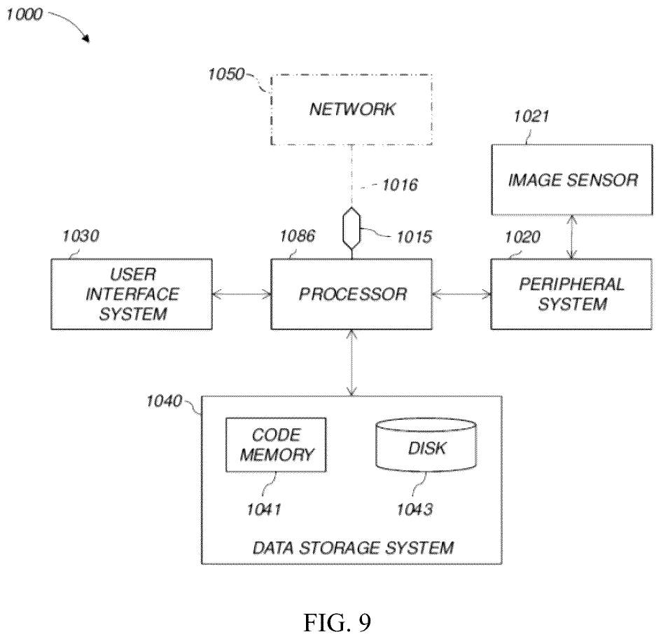

FIG. 9 is a high-level diagram showing the components of an exemplary data-processing system 1000 for analyzing data and performing other analyses described herein, and related components. The system includes a processor 1086, a peripheral system 1020, a user interface system 1030, and a data storage system 1040. The peripheral system 1020, the user interface system 1030 and the data storage system 1040 are communicatively connected to the processor 1086. Processor 1086 can be communicatively connected to network 1050 (shown in phantom), e.g., the Internet or a leased line, as discussed below. The data described above may be obtained using detector 1021 and/or displayed using display units (included in user interface system 1030) which can each include one or more of systems 1086, 1020, 1030, 1040, and can each connect to one or more network(s) 1050. Processor 1086, and other processing devices described herein, can each include one or more microprocessors, microcontrollers, field-programmable gate arrays (FPGAs), application-specific integrated circuits (ASICs), programmable logic devices (PLDs), programmable logic arrays (PLAs), programmable array logic devices (PALs), digital signal processors (DSPs), GPUs or cloud computing.

Processor 1086 which in one embodiment may be capable of real-time calculations (and in an alternative embodiment configured to perform calculations on a non-real-time basis and store the results of calculations for use later) can implement processes of various aspects described herein. Processor 1086 can be or include one or more device(s) for automatically operating on data, e.g., a central processing unit (CPU), microcontroller (MCU), desktop computer, laptop computer, mainframe computer, personal digital assistant, digital camera, cellular phone, smartphone, or any other device for processing data, managing data, or handling data, whether implemented with electrical, magnetic, optical, biological components, or otherwise. The phrase “communicatively connected” includes any type of connection, wired or wireless, for communicating data between devices or processors. These devices or processors can be located in physical proximity or not. For example, subsystems such as peripheral system 1020, user interface system 1030, and data storage system 1040 are shown separately from the data processing system 1086 but can be stored completely or partially within the data processing system 1086, including but not limited to digital signal processors (DSPs), GPUs or cloud computing.

The peripheral system 1020 can include one or more devices configured to provide digital content records to the processor 1086. For example, the peripheral system 1020 can include medical devices (such as medical imaging devices), digital still cameras, digital video cameras, cellular phones, or other data processors. The processor 1086, upon receipt of digital content records from a device in the peripheral system 1020, can store such digital content records in the data storage system 1040.

The user interface system 1030 can include a mouse, a keyboard, another computer (e.g., a tablet) connected, e.g., via a network or a null-modem cable, or any device or combination of devices from which data is input to the processor 1086. The user interface system 1030 also can include a display device, a processor-accessible memory, or any device or combination of devices to which data is output by the processor 1086. The user interface system 1030 and the data storage system 1040 can share a processor-accessible memory.

In various aspects, processor 1086 includes or is connected to communication interface 1015 that is coupled via network link 1016 (shown in phantom) to network 1050. For example, communication interface 1015 can include an integrated services digital network (ISDN) terminal adapter or a modem to communicate data via a telephone line; a network interface to communicate data via a local-area network (LAN), e.g., an Ethernet LAN, or wide-area network (WAN); or a radio to communicate data via a wireless link, e.g., WiFi or GSM. Communication interface 1015 sends and receives electrical, electromagnetic or optical signals that carry digital or analog data streams representing various types of information across network link 1016 to network 1050. Network link 1016 can be connected to network 1050 via a switch, gateway, hub, router, or other networking device.

Processor 1086 can send messages and receive data, including program code, through network 1050, network link 1016 and communication interface 1015. For example, a server can store requested code for an application program (e.g., a JAVA applet) on a tangible non-volatile computer-readable storage medium to which it is connected. The server can retrieve the code from the medium and transmit it through network 1050 to communication interface 1015. The received code can be executed by processor 1086 as it is received, or stored in data storage system 1040 for later execution.

Data storage system 1040 can include or be communicatively connected with one or more processor-accessible memories configured to store information. The memories can be, e.g., within a chassis or as parts of a distributed system. The phrase “processor-accessible memory” is intended to include any data storage device to or from which processor 1086 can transfer data (using appropriate components of peripheral system 1020), whether volatile or nonvolatile; removable or fixed; electronic, magnetic, optical, chemical, mechanical, or otherwise. Exemplary processor-accessible memories include but are not limited to: registers, floppy disks, hard disks, tapes, bar codes, Compact Discs, DVDs, read-only memories (ROM), Universal Serial Bus (USB) interface memory device, erasable programmable read-only memories (EPROM, EEPROM, or Flash), remotely accessible hard drives, and random-access memories (RAMs). One of the processor-accessible memories in the data storage system 1040 can be a tangible non-transitory computer-readable storage medium, i.e., a non-transitory device or article of manufacture that participates in storing instructions that can be provided to processor 1086 for execution.

In an example, data storage system 1040 includes code memory 1041, e.g., a RAM, and disk 1043, e.g., a tangible computer-readable rotational storage device such as a hard drive. Computer program instructions are read into code memory 1041 from disk 1043. Processor 1086 then executes one or more sequences of the computer program instructions loaded into code memory 1041, as a result performing process steps described herein. In this way, processor 1086 carries out a computer implemented process. For example, steps of methods described herein, blocks of the flowchart illustrations or block diagrams herein, and combinations of those, can be implemented by computer program instructions. Code memory 1041 can also store data, or can store only code.

Various aspects described herein may be embodied as systems or methods. Accordingly, various aspects herein may take the form of an entirely hardware aspect, an entirely software aspect (including firmware, resident software, micro-code, etc.), or an aspect combining software and hardware aspects. These aspects can all generally be referred to herein as a “service,” “circuit,” “circuitry,” “module,” or “system.”

Furthermore, various aspects herein may be embodied as computer program products including computer readable program code stored on a tangible non-transitory computer readable medium. Such a medium can be manufactured as is conventional for such articles, e.g., by pressing a CD-ROM. The program code includes computer program instructions that can be loaded into processor 1086 (and possibly also other processors) to cause functions, acts, or operational steps of various aspects herein to be performed by the processor 1086 (or other processor). Computer program code for carrying out operations for various aspects described herein may be written in any combination of one or more programming language(s), and can be loaded from disk 1043 into code memory 1041 for execution. The program code may execute, e.g., entirely on processor 1086, partly on processor 1086 and partly on a remote computer connected to network 1050, or entirely on the remote computer.

In certain embodiments, the systems and methods herein are particularly useful with any format for medical imaging. For example, the majority of reports produced by diagnostic medical imaging modalities are structured. The use of structured forms has been shown to reduce the ambiguity of natural language format reports and enhance the precision, clarity and value of clinical documents. At the technical level, a structured report (SR) is the optimal form of documentation in computerized systems as it allows searching, storage and comparison with similar data elements. Consequently, a Digital Imaging and Communications in Medicine (DICOM) SR has emerged to increase the efficiency of the distribution of information between various specialties such as computed tomography (CT), magnetic resonance imaging (MRI), ultrasound, etc.

The DICOM SR is a document architecture designed for encoding and exchanging information using the DICOM hierarchical structure and services. For example, the DICOM SR introduces DICOM information object definitions (IODs) and services used for the storage and transmission of SRs. The DICOM IODs define data structures that describe information objects of real-world objects (e.g., patients, images and reports) that are involved, for example, in radiology operations. The DICOM services are concerned with storage, query, retrieval and transfer of data.

An exemplary DICOM SR consists of a sequence of nodes called “Content Items” that are linked together via relationships. Several exemplary relationships defined by DICOM are: ‘HAS OBS CONTEXT’ where the target conveys an observation context for documentation of the source; ‘CONTAINS’ where the source contains the target; ‘HAS CONCEPT MOD’ which qualifies or describes the concept name of the source; ‘HAS PROPERTIES’ which is a description of properties of the source; ‘HAS ACQ CONTEXT’ where the target describes the condition during data acquisition of the source; ‘SELECTED FROM’ where the source conveys spatial or temporal coordinates selected from the target; and ‘INFERRED FROM’ where the source conveys a measurement or other inference made from the target.

Each content item is represented by a name/value pair. The name refers more precisely to a “Concept Name”, which is defined by a code rather than by free text to facilitate indexing and searching. Any concept name may be represented by a coded entry that uses the following triplet encoding attributes: ‘Code Value’ which is a computer-readable and -searchable identifier, ‘Code Scheme Designator’ which is an identifier of the coding organization and ‘Code Meaning’ in which human-readable text is entered to be displayed.

The value of a content item is typically one of the following: ‘CONTAINER’ for headings or categories; ‘TEXT’ for free form textual expression; ‘PNAME’ for a patient's name; ‘DATETIME’ which is a concentrated date and time of occurrence; ‘DATE’ which is the calendar date of occurrence; ‘TIME’ which is time of day of occurrence; ‘NUM’ for numeric values or measurements with associated units; ‘IMAGE’ for unique identifier (UID) references to image service-object-pair (SOP) instances; ‘WAVEFORM’ for UID references to waveform SOP instances; ‘COMPOSITE’ for UID references to composite SOP instances; ‘UIDREF’ for UIDs identified by concept name; ‘SCOORD’ for spatial coordinates of a geometric region of interest (ROI) in images; ‘TCOORD’ for temporal coordinates of an ROI in waveforms; and ‘CODE’ which is a coded expression of the concept. A parent content item (e.g., source node) can be linked to a child content item (e.g., target node) with one of the relationships just described.

Today, the DICOM SR has become a powerful format that improves the expressiveness, precision and comparability of clinical documentation. For example, the DICOM SR provides the capability to link a clinical document to DICOM images and waveforms such that they can be displayed simultaneously at the same workstation. Further, the DICOM SR is a “databaseable document” format that facilitates computer search analysis for various purposes, such as scientific research, education, training, clinical trials, performance evaluation, and eventually for integration with data mining applications.

### INCORPORATION BY REFERENCE

References and citations to other documents, such as patents, patent applications, patent publications, journals, books, papers, web contents, have been made throughout this disclosure, including to the Supplementary. The Supplementary, and all other such documents are hereby incorporated herein by reference in their entirety for all purposes.

### Equivalents

The invention may be embodied in other specific forms without departing from the spirit or essential characteristics thereof. The foregoing embodiments are therefore to be considered in all respects illustrative rather than limiting on the invention described herein.

### EXAMPLES

### Example 1

### Pressure-Gradient Induced Velocity-Gradient Correction (PG-VGC)

The WSS is potentially associated with the physiological remodeling of the blood vessel wall, thus provides clinical indication on the growth and rupture of the intracranial cerebral aneurysms, aortic dilatation, etc. In vivo 4D flow MRI provides time-resolved 3D measurement of velocity fields within the cardiovascular system, thus enables the evaluation of the local and instantaneous WSS.

Under prior art approaches, WSS calculation from velocity gradient tensor (Vgrads):

τ̅=μ(∇u+∇u^{T}),
with wall normal vector n̅ (pointing inwards): τ̅=τ̅·n̅
and WSS magnitude: τ_{mag}=|τ̅|.
   Additionally, WSS calculation from 1D velocity gradient obtained by grid rotation, interpolation, and smooth 1D spline fitting (Spline):
Local coordinate system rotation: R̅·[x, y, z]=[x′, y′, z′]
Find velocity on by interpolation and rotation: u̅′̅=R̅·ū

$$WSS : \overline{{τ}^{′}} = μ [ \frac{\partial {u}^{′}}{\partial {z}^{′}} , \frac{\partial {v}^{′}}{\partial {z}^{′}} , 0 ] , \overline{τ} = {\overline{\overline{R}}}^{- 1} \cdot \overline{{τ}^{′}} .$$

Under these prior art techniques, the limited spatial resolution and measurement noise of 4D flow MRI can affect the reliability of the obtained WSS.

FIG. 1 provides the method flow chart of the proposed pressure-gradient induced velocity-gradient correction (PG-VGC). The method provides a novel WSS estimation method for 4D flow MRI. The method uses the pressure gradient estimated from the flow data in the whole ROI and flow physics constraints to correct the velocity gradient, therefore enhancing the WSS estimation.

The pressure field was reconstructed using WLS with the weights given as a function of the distance to the wall:

$$w ( {s}_{wall} ) = {w}_{min} + ( {w}_{max} - {w}_{min} ) \frac{{s}_{wall}}{{s}_{wall , max}} , with \frac{{w}_{max}}{{w}_{min}} = 10 ,$$

where s_{wall} means the distance from the wall.
The velocity gradients were enhanced by subtracting the velocity gradient errors (∇u=∇uₜ+ϵ_{∇u}) estimated from:

$$COLM \text{:} \frac{{∇}_{p}}{ρ} = - \frac{\partial u}{\partial t} - u \cdot ( ∇ u - {ϵ}_{{∇}_{u}} ) + v ∇ \cdot ( ∇ u - {ϵ}_{{∇}_{u}} )$$

$$COM : e \cdot ( ∇ u - {ϵ}_{{∇}_{u}} ) = 0.$$

An error analysis with synthetic MRI data was then performed using the methods and systems as described herein. Synthetic MRI data were generated from analytical Womersley flow.
Womersley number: 1, 2, 4, 8, 12, 16
Spatial resolution (D/h): 2, 3, 4, 5, 7, 9, 11, 14
Noise level (1/VNR): 0%, 10%
   The relative RMSE of WSS estimated from synthetic Womersley 4D flow datasets with 0% noise. See FIG. 2 panels A-C and FIG. 3 panels A-B.

Compared to Spline and Vgrads, the proposed PG-VGC method yielded more accurate WSS for most datasets. Greater improvement was achieved by PG-VGC for higher α cases with more than 100% improvement at α of 12 and 16.

An analysis with synthetic aneurysmal flow was then conducted. Synthetic MRI data were generated from the patient-specific CFD simulations of a basilar tip (BT) aneurysm and an internal carotid artery (ICA) aneurysm. WSS results were evaluated from synthetic MRI data using the proposed method (PG-VGC) and the existing state-of-the-art methods (Spline and Vgrads) and were compared to CFD (ground truth). The Bland-Altman plots (FIG. 5 panel A) and the spatial distributions of the TAWSS (FIG. 5 panel B) showed that the proposed PG-VGC method estimated more accurate WSS than the existing methods.

The methods and systems of the invention were then applied to in vivo aneurysmal 4D flow data. As shown in FIG. 6 panels A-C, the increased WSS predicted by PG-VGC improved the accuracy of the WSS estimation in cerebral aneurysms. PG-VGC predicted the high-WSS regions which were not captured using Vgrads or Spline.

The methods and systems of the invention were then applied to in vivo aortic 4D flow. Data. As shown in FIG. 7 panels A-B, the PG-VGC predicted 4 to 6 times higher mean WSS than Spline and Vgrads resulting in better agreement with the results from previous CFD studies.

The methods and systems of the invention were then applied to determine spatial distributions of aortic WSS. As shown in FIG. 8 panels A-B, the high WSS regions predicted by PV-VGC showed improved correlation with vortical structures (circles).

Accordingly, the data herein show that the invention provides a novel WSS estimation method for 4D flow MRI. The method uses the pressure gradient estimated from the flow data in the whole ROI and flow physics constraints to correct the velocity gradient, therefore enhancing the WSS estimation. The method's performance was evaluated using synthetic and in vivo 4D flow data in cerebral aneurysms and thoracic aortas. The proposed method showed reliable estimation of the mean and the relative distribution of WSS with as much as 100% improvement in WSS accuracy. The data herein shows that the systems and methods of the invention can benefit clinical applications of 4D flow MRI as it improves the accuracy of the WSS estimation.

What is claimed is:

1. A method for determining Wall Shear Stress (WSS) with 4D flow Magnetic Resonance Imaging (MRI), the method comprising:
receiving 4D MRI flow data;
calculating a velocity gradient from the 4D MRI flow data;
correcting the velocity gradient, thereby producing a corrected velocity gradient; and
determining a WSS from the corrected velocity gradient.

2. The method of claim 1, wherein the method further comprises calculating a pressure field and using a spatial gradient of the pressure field to correct the velocity gradient, wherein the spatial gradient of the pressure field is employed to correct the velocity gradient based on the conservation of mass (COM) and conservation of linear momentum (COLM).

3. The method of claim 2, wherein the pressure field was calculated using a weighted approach with weights given as a function of physical and measurement variables:
w(s_wall)=w_min+(w_max−w_min) s_wall/s_(wall,max), with w_max/w_min=10;
where s_wall means distance from the blood vessel wall.

4. The method of claim 2, wherein the corrected velocity gradient was determined by subtracting velocity gradient errors (∇u=∇u_t+ϵ_∇u) estimated from:
COLM: ∇p/ρ=−∂u/∂t−u·(∇u−ϵ_∇u)+v∇·(∇u−ϵ_∇u)
COM: e·(∇u−ϵ_∇u)=0.

5. The method of claim 2, wherein the method uses the 4D MRI flow data in a whole region of interest (ROI).

6. The method of claim 5, wherein the pressure field is reconstructed by integrating a pressure gradient estimated from a velocity and the velocity gradient in the whole ROI.

7. The method of claim 6, wherein the reconstructed pressure field gradient is employed with additional regularization from a divergence-free constraint to correct the velocity gradient.

8. The method of claim 7, wherein the WSS is estimated based on the corrected near-wall velocity gradient.

9. The method of claim 1, wherein the WSS is used to analyze physiological remodeling of a blood vessel wall.

10. The method of claim 9, wherein results of the analysis of the physiological remodeling of a blood vessel wall provides an indication on growth and/or rupture of the blood vessel wall.

11. A system for determining Wall Shear Stress (WSS) with 4D flow Magnetic Resonance Imaging (MRI), the system comprising a processor configured to:
receive 4D MRI flow data;
calculate a velocity gradient from the 4D MRI flow data;
correct the velocity gradient, thereby producing a corrected velocity gradient; and
determine a WSS from the corrected velocity gradient.

12. The system of claim 11, wherein the method further comprises calculating a pressure field and using a spatial gradient of the pressure field to correct the velocity gradient, wherein the spatial gradient of the pressure field is employed to correct the velocity gradient based on the conservation of mass (COM) and conservation of linear momentum (COLM).

13. The system of claim 12, wherein the pressure field was calculated using a weighted approach with weights given as a function of physical and measurement variables:
w(s_wall)=w_min+(w_max−w_min) s_wall/s_(wall,max), with w_max/w_min=10;
where s_wall means distance from the blood vessel wall.

14. The system of claim 12, wherein the corrected velocity gradient was determined by subtracting velocity gradient errors (∇u=∇u_t+ϵ_∇u) estimated from:
COLM: ∇p/ρ=−∂u/∂t−u·(∇u−ϵ_∇u)+v∇·(∇u−ϵ_∇u)
COM: e·(∇u−ϵ_∇u)=0.

15. The system of claim 12, wherein the processor uses the 4D MRI flow data in a whole region of interest (ROI).

16. The system of claim 15, wherein the pressure field is reconstructed by integrating a pressure gradient estimated from a velocity and the velocity gradient in the whole ROI.

17. The system of claim 16, wherein the reconstructed pressure field gradient is employed with additional regularization from a divergence-free constraint to correct the velocity gradient.

18. The system of claim 17, wherein the WSS is estimated based on the corrected near-wall velocity gradient.

19. The system of claim 11, wherein the WSS is used to analyze physiological remodeling of a blood vessel wall.

20. The system of claim 19, wherein results of the analysis of the physiological remodeling of a blood vessel wall provides an indication on growth and/or rupture of the blood vessel wall.
